SEQUENCE LISTING (8 sequences)

SEQ ID NO: 1 - DNA, 114 bp, Homo sapiens
cactcggacgggatcttcacggacagctacagccgctaccggaaacaaatggctgtcaagaaatacttggcggccgtcctagggaagaggtataaacaaagggttaaaaacaaa

SEQ ID NO: 2 - protein, 38 aa, Homo sapiens
HSDGIFTDSYSRYRKQMAVKKYLAAVLGKRYKQRVKNK

SEQ ID NO: 3 - protein, 525 aa, Homo sapiens
MAGVVHVSLAAHCGACPWGRGRLRKGRAACKSAAQRHIGADLPLLSVGGQWCWPRSVMAGVVHVSLAALLLLPMAPAMHSDCIFKKEQAMCLEKIQRANELMGFNDSSPGCPGMWDNITCWKPAHVGEMVLVSCPELFRIFNPDQVWETETIGESDFGDSNSLDLSDMGVVSRNCTEDGWSEPFPHYFDACGFDEYESETGDQDYYYLSVKALYTVGYSTSLVTLTTAMVILCRFRKLHCTRNFIHMNLFVSFMLRAISVFIKDWILYAEQDSNHCFISTVECKAVMVFFHYCVVSNYFWLFIEGLYLFTLLVETFFPERRYFYWYTIIGWGTPTVCVTVWATLRLYFDDTGCWDMNDSTALWWVIKGPVVGSIMVNFVLFIGIIVILVQKLQSPDMGGNESSIYLRLARSTLLLIPLFGIHYTVFAFSPENVSKRERLVFELGLGSFQGFVVAVLYCFLNGEVQAEIKRKWRSWKVNRYFAVDFKHRHPSLASSGVNGGTQLSILSKSSSQIRMSGLPADNLAT

SEQ ID NO: 4 - protein, 33 aa, Artificial sequence
  PACAP with first 5 amino acids truncated
FTDSYSRYRKQMAVKKYLAAVLGKRYKQRVKNK

SEQ ID NO: 5 - protein, 44 aa, Artificial Sequence
  Sand fly - truncation of SEQ.ID.NO.6 Maxadilan
CDATCQFRKAIDDCQKQAHHSNVPGNSVFKECMKQKKKEFKAGK

SEQ ID NO: 6 - protein, 61 aa, Sand fly [sequence omitted]

SEQ ID NO: 7 - protein, 27 aa, Homo sapiens
HSDGIFTDSYSRYRKQMAVKKYLAAVL

SEQ ID NO: 8 - protein, 28 aa, Homo sapiens
HSDAVFTDNYTRLRKQMAVKKYLNSILN

(19) United States
(12) Patent Application Publication
(10) Pub. No.: US 2002/0182729 A1
(43) Pub. Date: Dec. 5, 2002

(54) Pituitary adenylate cyclase-activating polypeptide (PACAP) is an anti-mitogenic signal for selected neuronal precursors in vivo

(76) Inventors: Emanuel DiCicco-Bloom, Princeton Junction, NJ (US); Arnaud Nicot, New Brunswick, NJ (US); Nairu Lu, Jersey City, NJ (US); Junghyup Suh, Piscataway, NJ (US)

Correspondence Address: LYON & LYON LLP, 633 WEST FIFTH STREET, SUITE 4700, LOS ANGELES, CA, 90071, US

(21) Appl. No.: 10/044,722

(22) Filed: Jan. 11, 2002

Related U.S. Application Data
(60) Provisional application No. 60/260,909, filed on Jan. 11, 2001.

(51) Int. Cl. C12N 5/08

(52) U.S. Cl. 435/368

(57) ABSTRACT

Pituitary adenylate cyclase-activating polypeptide (PACAP) and its receptor system can be manipulated positively or negatively to regulate mitosis in neuronal precursor cells. The ligand/receptor system involves PACAP, PACAP receptor, PAC,, and related antagonists. The methods of regulation of the present invention may model be used to define cell cycle regulation in the developing neurons. The present invention may also be used to control or cure diseases related to or caused by damage to or destruction of neuronal cells.

### CROSS-REFERENCE TO RELATED APPLICATIONS

The present utility patent application claims priority from US Provisional Application Serial No. 60/260,909 (Di-Cicco-Bloom, et al.), filed Jan. 11, 2001, the disclosure of which is incorporated by reference in its entirety herein.

### GOVERNMENT INTEREST

This work was supported in part by National Institutes of Health Grant R01 NS32401. This invention was made with government support. The government may own certain rights in the present invention.

### TECHNICAL FIELD

The present invention relates to the field of developmental and regenerative neurology. Specifically, the present invention relates to a method of mitotic regulation of neuronal precursors characterized by the use of a ligand/receptor system of pituitary adenylate cyclase-activating polypeptide (PACAP), PACAP's receptor, PAC1, and related agonists and antagonists.

### BACKGROUND OF THE INVENTION

Various publications or patents are referred to in textual citations throughout this application to describe the state of the art to which the invention pertains. Each of these publications or patents is incorporated by reference herein. Complete citations of scientific publications are set forth at the end of the specification.

The developing forebrain contains several types of cells, including cerebral cortical precursor cells, subventricular zone precursor cells, hippocampal granule precursor cells, and various types of glial precursor cells. Not all precursor cells types are present throughout the entire embryonic development of an animal. The cerebral cortex is the extensive outer layer of unmyelinated neurons of the cerebral hemispheres. The cerebral cortex is largely responsible for higher brain functions, including sensation, voluntary muscle movement, thought, reason, and memory. Because the cerebral cortex, and the forebrain in general, is involved in so many important functions for humans, diseases or disorders affecting the cerebral cortex are varied and can be quite serious or even deadly. Examples of neurodegenerative diseases or disorders involving the neurons of the forebrain include stroke, dementia, primary cortical degenerative disorders, sub-cortical degenerative disorders, infections, prion disorders, toxic and metabolic disorders, and brain injury.

In a developing forebrain of animals, precise control of proliferation is required because the size of the neuronal precursor population determines the number of neurons generated, regulating the final size of the cortex (Takahashi, T 1996). Some factors stimulating proliferation have been defined. During development, neurogenesis, which is the differentiation of the nervous system from the ectoderm of the early embryo, depends on neuronal precursor proliferation and terminal withdrawal from the cell cycle according to well-characterized schedules. An important factor controlling neuronal precursor cell proliferation in the forebrain on all forebrain cell types discussed herein is PACAP (Pituitary Adenylate Cyclase-Activating Polypeptide).

PACAP, the 38-residue neuropeptide, was discovered in the late 1980's as a hypothalamic neuropeptide showing highest homology with VIP (Vasoactive Intestinal Polypeptide) of secretin/glucagon family (Miyata, 1989). PACAP interacts via three G-protein coupled receptors, VPAC 1, VPAC 2, and PAC₁. The PACAP-specific receptor, PAC₁, is a seven transmembrane G-protein coupled receptor. PAC₁ is expressed, among other locations, in the central and peripheral nervous system, in the pituitary gland, in adrenal glands and in ovaries. VPAC 1 and VPAC 2 have high affinity for both VIP and PACAP, whereas PAC₁ binds only to PACAP with high affinity. Activation of the three PACAP receptors typically leads to a robust Gs-mediated cAMP elevation, while PAC₁ receptors can also link to other transduction pathways such as phospholipase C (PLC) and calcium mobilization (Arimura, 1998). These effects have been linked to different PAC₁ splice isoforms (Spengler et al., 1993; Chatteijee et al., 1996; Nicot and DiCicco-Bloom, 2001).

Previously, PACAP has been defined as an autocrine inhibitor of cortical precursor mitosis in culture (Lu, N 1997). PACAP ligand/receptor system is expressed during peripheral (Di-Cicco-Bloom, E 2000; Waschek, J A 1998) and central neurogenesis (Waschek, J A 1998; Tatsuno, I 1994; Sheward, W J 1996), and elicits opposing mitogenic regulation in vitro according to neural lineage. Several mitogens and receptors, including bFGF and IGF-1, are expressed, and the factors stimulate proliferation of cortical precursors in vitro (Drago J, 1991; Ghosh A, 1995) and in vivo (Vaccarino, F M 1999). However, ontogenetic functions relating to the PACAP ligand/receptor system in the cortex of the living embryo remain undefined. While diminished mitogen activity might control the sequential cell cycle exit producing post-mitotic neurons, precursor proliferation may alternatively be subject to negative regulation.

Due to the uncertainty of the effects of PACAP in vivo, it would be advantageous to determine if certain factors' sustained expression throughout neurogenesis suggest that additional signals may be required to directly inhibit mitosis. (Drago J, 1991; Ghosh A, 1995; Vaccarino, F M 1999). It would be a significant improvement in the art if the neuronal cells could be generated (positively regulated) or suppressed (negatively regulated) based upon the interactions of PACAP, PACAP receptor, PAC₁, and antagonists and agonists in the ligand/receptor systems of the neural cells. The advantages of regenerating neurons in the adult brain include re-growing brain tissue after any event that causes cerebral degeneration, damage or destruction. Finally, there could be advantages in preventing the growth of neurons or neuronal precursors, such as when the neurons, including glial and other neurons, are implicated in an uncontrolled proliferative disease like cancer.

### SUMMARY OF THE INVENTION

The present invention provides methods for using PACAP, PACAP receptor, PAC₁ , and related antagonists and agonists to proliferate neuronal precursor cells.

The present invention further relates to methods of using PACAP, the PAC₁ receptor, and related antagonists and agonists for controlling neuronal precursor cell growth.

A further embodiment of the present invention relates to using mRNA and antibodies to manipulate the expression of PACAP within neuronal cells, thus promoting the proliferation of the manipulated cells.

In another aspect of the present invention, methods for controlling the proliferation of neuronal cells using PACAP to prevent the diseases or disorders are elucidated. For medical conditions caused by the proliferation of cerebral cortical cells, subventricular zone cells, or hippocampal granule or glial cells, PACAP is administered to cells. For medical conditions caused or exacerbated by a lack of cerebral cortical precursor cells, subventricular zone precursor cells, or hippocampal granule or glial cells, an antagonist for a PACAP receptor is administered to the cells. Because all cerebral cortical cells, subventricular zone cells, or hippocampal granule or glial cells are part of the forebrain and have the PACAP receptor/ligand system, the present invention directs the methods of the present invention to all referenced precursor cell types.

### BRIEF DESCRIPTION OF THE DRAWINGS

FIG. 1, parts (a-e) are representations of the PAC₁ receptor expressed in the developing E15.5 rat cortex.

FIG. 1, parts (f-h) are representations of PACAP expressed in embryonic cortex.

FIG. 1, parts (i-m) are representations of proliferative precursors expressing PACAP and PAC₁ receptors.

FIG. 2 shows representations of intraventricular PACAP inhibiting precursor mitosis and neurogenesis without enhancing cell death.

FIG. 3 is a listing of the amino acid sequences for PACAP, PAC1 and related antagonists and agonists.

### DETAILED DESCRIPTION OF THE INVENTION

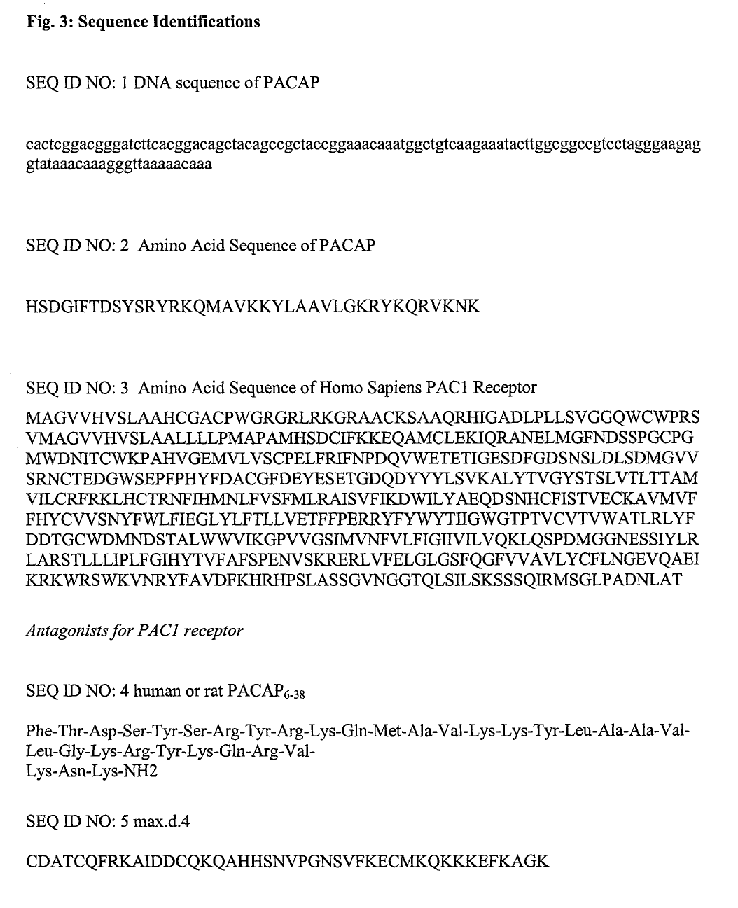

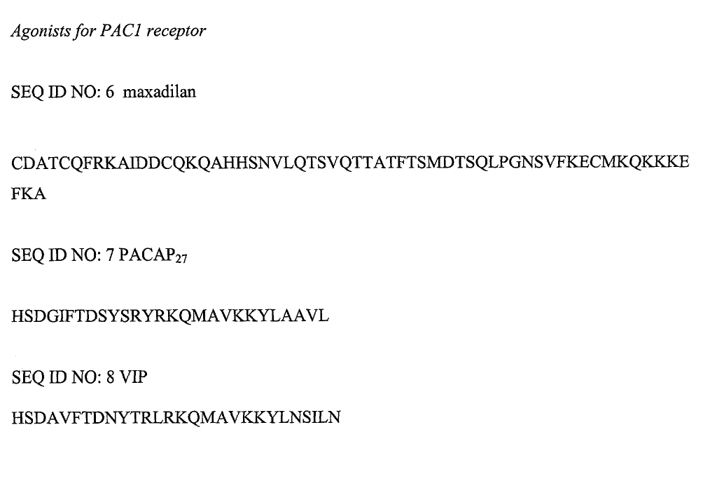

The present invention relates to the use of a neuropeptide, PACAP identified in SEQ. ID. NO: 1 (DNA sequence) and SEQ. ID. NO: 2 (amino acid sequence) for controlling mitosis in neurons, specifically cerebral cortical neurons. (All “SEQ. ID. NO:” sequences are referenced in FIG. 3.) The present invention further relates to PACAP's receptor, PAC₁ (SEQ. ID. NO: 3), and related antagonists and agonists. For example, known antagonists of the PACAP receptor include PACAP₆₋₃₈ (SEQ. ID. NO: 4) and max. d. 4 (SEQ. ID. NO: 5). Known agonists of the PACAP receptor include maxadilan (SEQ. ID. NO: 6), PACAP₂₇ (SEQ. ID. NO: 7), and VIP (SEQ. ID. NO: 8).

The PACAP ligand/receptor system and mitotic inhibition mediated by endogenous peptide and the cAMP pathways in vivo show that anti-mitogenic signals actively restrain growth factor-induced proliferation of precursors during brain ontogeny. Further, because some neurons are now known to regenerate in adult brains under certain conditions, the present invention also may be used to the control of neuronal growth in adults. Thus, manipulating the PACAP ligand/receptor system to affect mitosis in selected neuronal precursors allows for control over the proliferation of those cells. This use of the PACAP (SEQ. ID. NO: 2) ligand/receptor system may also be used for neurons that utilize PACAP in the same or a similar way to control mitosis as the forebrain neuronal cells do. The control can be exercised, either by proliferating the neuronal cells or by limiting or ceasing proliferation of the neuronal cells. By controlling the amount of PACAP in the cells, and correspondingly, the production of endogenous PACAP, external control may be exercised over mitosis of cerebral cortical neurons. Controlling the genesis and number of neurons has wide-ranging scientific and therapeutic application as will be seen in the following embodiments.

One embodiment of the invention relates to proliferating neuronal precursor cells. Because PACAP, administered in vivo, is an anti-mitogenic signal, an antagonist to the PACAP receptor PAC₁ (SEQ. ID. NO: 3) may be used. An antagonist will prevent the PACAP from binding to its receptor. Thus, PACAP will not be able to stop mitosis in the G1-S phase and the cells will continue with their maturity in the cell cycle. Some PACAP antagonists that may be used are PACAP₆₋₃₈ (SEQ. ID. NO: 4) and max. d. 4 (SEQ. ID. NO: 5).

Another embodiment of promoting proliferation of neuronal cells found in the forebrain is to provide an oligonucleotide consisting of a sequence complementary to PACAP and introducing the oligonucleotide into the cells. This embodiment allows for the expression of endogenous PACAP in the cells to be decreased, thus resulting in the continued proliferation of the neuronal cells.

In practice, this embodiment may be achieved by the use of the recombinant PACAP DNA, cDNA, RNA or polynucleotide sequences coding for the antisense sequence encoding the protein, may be delivered to the neuronal precursor cells for the production or inhibition of PACAP endogenously, by use of biologically compatible carriers or excipients. This may be useful in inducing or inhibiting cerebral cortical precursor proliferation and/or differentiation. Pharmaceutically acceptable carriers for therapeutic use are well known in the pharmaceutical art, and are described, for example, in Remington's Pharmaceutical Sciences (A. P. Gennaro, ed.; Mack, 1985). For example, sterile saline or phosphate-buffered saline at physiological pH may be used. Preservatives, stabilizers, dyes, and even flavoring agents may be provided in the pharmaceutical composition. For example, sodium benzoate, sorbic acid, and esters of p-hydroxybenzoic acid may be added as preservatives. Antioxidants and suspending agents may also be used.

The pharmaceutical compositions of the present invention may be formulated and used as tablets, capsules, or elixirs for oral administration; sterile solutions and suspensions for parenteral administration; aerosols or insufflations for intratracheobronchial administration; and the like. PACAP is known to pass through the blood-brain barrier and this property should be considered when determining the most advantageous route of administration. Preparations of such formulations are well known to those skilled in the pharmaceutical arts. The dosage and method of administration can be tailored to achieve optimal efficacy and will depend on factors that those skilled in the medical arts will recognize.

When administration is to be parenteral, such as intravenous on a daily basis, injectable pharmaceuticals may be prepared in conventional forms, either as liquid solutions or suspensions; solid forms suitable for solution or suspension in liquid prior to injection; or as emulsions. Suitable excipients are, for example, water, saline, dextrose, mannitol, lactose, lecithin, albumin, sodium glutamate, cysteine hydrochloride, or the like. In addition, if desired, the injectable pharmaceutical compositions may contain minor amounts of nontoxic auxiliary substances, such as wetting agents, pH buffering agents, and the like. If desired, absorption enhancing preparations (e.g. liposomes) may be utilized.

Hence, a preferred embodiment the present invention is directed to novel pharmaceutical compositions that include a biologically acceptable carrier along with an effective amount of a PACAP polypeptide, PAC₁ agonist or PAC₁ antagonist or PACAP DNA, cDNA or RNA for manipulating the mitotic division of neuronal precursor cells.

In one particular embodiment the pharmaceutical composition includes a PACAP polypeptide sequence substantially identical to SEQ. ID. NO: 2 and/or the polynucleotide sequence that is substantially similar to SEQ ID NO: 1 and encodes a PACAP polypeptide. In a further embodiment, the pharmaceutical composition includes an antagonist amino acid sequence substantially identical to SEQ. ID. NO: 4 or SEQ. ID. NO: 5. In yet another embodiment, the pharmaceutical composition includes an agonist amino acid sequence substantially identical to SEQ. ID. NO: 6, SEQ. ID. NO: 7 or SEQ. ID. NO: 8.

Another preferred embodiment for the use of the PACAP ligand receptor system to suppress the activity of PACAP, and thus promote neuronal precursor proliferation involves the use of antibodies. In this embodiment, antibodies that bind to PACAP and cells comprising PACAP are provided, and the antibody is introduced into or contacted with the cells. The method teaches using antibodies capable of immunospecifically binding to PACAP. The antibody decreases the expression of PACAP in the cells.

Polyclonal or monoclonal antibodies directed towards PACAP may be prepared according to standard methods. Monoclonal antibodies may be prepared according to general hybridoma methods of Kohler and Milstein, (1975) the trioma technique, the human B-cell hybridoma technique (Kozbor et al., 1983) and the EBV-hybridoma technique (Cole et al., 1985).

Antibodies utilized in the present invention may be polyclonal antibodies, although monoclonal antibodies are preferred -because they may be reproduced by cell culture or recombinantly, and may be modified to reduce their antigenicity.

Polyclonal antibodies may be raised by a standard protocol by injecting a production animal with an antigenic composition, formulated as described above. (Harlow and Lane, 1988). In one such technique, a PACAP antigen comprising an antigenic portion of the PACAP protein is initially injected into any of a wide variety of mammals (e.g., mice, rats, rabbits, sheep or goats). Alternatively, an immune response may be elicited if the polypeptide is joined to a carrier protein, such as ovalbumin, BSA or KLH. The peptide-conjugate is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line. The immortal cell line utilized is preferably selected to be deficient in enzymes necessary for the utilization of certain nutrients. Many such cell lines (such as myelomas) are known to those skilled in the art, and include, for example: thymidine kinase (TK) or hypoxanthine-guanine phosphoriboxyl transferase (HGPRT). These deficiencies allow selection for fused cells according to their ability to grow on, for example, hypoxanthine aminopterinthymidine medium (HAT).

Preferably, the immortal fusion partners utilized are derived from a line that does not secrete immunoglobulin. The resulting fused cells, or hybridomas, are cultured under conditions that allow for the survival of fused, but not unfused, cells and the resulting colonies screened for the production of the desired monoclonal antibodies. Colonies producing such antibodies are cloned, expanded, and grown so as to produce large quantities of antibody (Kohler and Milstein, 1975).

Large quantities of monoclonal antibodies from the secreting hybridomas may then be produced by injecting the clones into the peritoneal cavity of mice and harvesting the ascites fluid therefrom. The mice, preferably primed with pristine, or some other tumor-promoter, and immunosuppressed chemically or by irradiation, may be any of various suitable strains known to those in the art. The ascites fluid is harvested from the mice and the monoclonal antibody purified therefrom, for example, by CM Sepharose column or other chromatographic means. Alternatively, the hybridomas may be cultured in vitro or as suspension cultures. Batch, continuous culture, or other suitable culture processes may be utilized. Monoclonal antibodies are then recovered from the culture medium or supernatant.

In addition, the antibodies or antigen binding fragments may be produced by genetic engineering. In this technique, as with the standard hybridoma procedure, antibody-producing cells are sensitized to the desired antigen or immunogen. The messenger RNA isolated from the immune spleen cells or hybridomas may be used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones that co-express a heavy and light chain (resembling the Fab fragment or antigen binding fragment of an antibody molecule). The vectors that carry these genes are co-transfected into a host (e.g. bacteria, insect cells, mammalian cells, or other suitable protein production host cell.). When antibody gene synthesis is induced in the transfected host, the heavy and light chain proteins self-assemble to produce active antibodies that can be detected by screening with the antigen or immunogen.

Chimeric antibodies may be made by recombinant means by combining the murine variable light and heavy chain regions (VK and VH), obtained from a murine (or other animal-derived) hybridoma clone, with the human constant light and heavy chain regions, in order to produce an antibody with predominantly human domains. The production of such chimeric antibodies is well known in the art, and may be achieved by standard means (as described, e.g., in U.S. Pat. No. 5,624,659, incorporated fully herein by reference). Humanized antibodies are engineered to contain even more human-like immunoglobulin domains, and incorporate only the complementarity-determining regions of the animal-derived antibody. This is accomplished by carefully examining the sequence of the hyper-variable loops of the variable regions of the monoclonal antibody, and fitting them to the structure of the human antibody chains. Although facially complex, the process is straightforward in practice. See, e.g., U.S. Pat. No. 6,187,287, incorporated fully herein by reference.

In a preferred embodiment, antibodies are prepared that react immunospecifically with various epitopes of the PACAP encoded protein. These above-described antibodies may be employed to bind to the endogenous PACAP protein on a cell's surface membrane and thereby prevent it from binding to the PAC₁ receptor. Hence, these antibodies may be used in pharmaceutical compositions for therapeutic purposes where it is desirable to proliferate neuronal precursor cells. Specific antibodies may be made in vivo using recombinant DNA and methods well know in the art.

For administration, the antibody-therapeutic agent will generally be mixed, prior to administration, with a non-toxic, pharmaceutically acceptable carrier substance. Usually, this will be an aqueous solution, such as normal saline or phosphate-buffered saline (PBS), Ringer's solution, lactate-Ringer's solution, or any isotonic physiologically acceptable solution for administration by the chosen means. Preferably, the solution is sterile and pyrogen-free, and is manufactured and packaged under current Good Manufacturing Processes (GMP's), as approved by the FDA. The clinician of ordinary skill is familiar with appropriate ranges for pH, tonicity, and additives or preservatives when formulating pharmaceutical compositions for administration by intravascular injection, intrathecal injection, or by other routes. In addition to additives for adjusting pH or tonicity, the antibody-therapeutics agent may be stabilized against aggregation and polymerization with amino acids and non-ionic detergents, polysorbate, and polyethylene glycol.

Optionally, additional stabilizers may include various physiologically acceptable carbohydrates and salts. Also, polyvinylpyrrolidone may be added in addition to the amino acid. Suitable therapeutic immunoglobulin solutions, which are stabilized for storage and administration to humans, are described in U.S. Pat. No. 5,945,098, incorporated fully herein by reference. Other agents, such as human serum albumin (HSA), may be added to the therapeutic composition to stabilize the antibody conjugates. The compositions of the invention may be administered using any medically appropriate procedure.

The effective amount of the therapeutic antibody composition to be given to a particular patient will depend on a variety of factors, several of which will be different from patient to patient. A competent clinician should be able to determine an effective amount of a therapeutic antibody-conjugate composition to administer to a patient. Dosage of the antibody-therapeutic will depend on the type of treatment previously administered, route of administration, the nature of the therapeutics, sensitivity of the stem cells to the therapeutics, etc. Utilizing LD₅₀ animal data, and other information available, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions, which are rapidly cleared from the body, may be administered at higher doses, or in repeated doses, in order to maintain a therapeutic concentration. Utilizing ordinary skill, the competent clinician will be able to optimize the dosage of a particular therapeutic composition in the course of routine clinical trials.

The various embodiments of the invention that contemplate inhibiting the production of PACAP and lessening the presence of PACAP in the neuronal precursor cells and thus, stopping PACAP from preventing mitosis, may be used for a variety of therapeutic uses, as described above. A number of diseases involve neuronal cells found in the forebrain, including stroke, dementia, primary cortical degenerative disorders, sub-cortical degenerative disorders, infections, prion disorders, toxic and metabolic disorders, and brain injury. The various methods of using the present invention in a research or clinical setting may potentially encourage the regrowth of neuronal cells in adult brains. Finally, because of the experiments contained herein, the various methods of the present invention could also be used on embryos or on neonatal babies with neuronal disorders.

In yet another aspect of the present invention, the PACAP ligand/receptor system is used to inhibit the proliferation of neuronal precursor cells. This result may be achieved, either by increasing the PACAP in the cells, by introducing agonists for PACAP into the cells, or both. For example, using multiple agonists may increase the inhibitory effect because the ligands may saturate the receptors more effectively or at a faster rate. In the embodiment that requires the introduction of a composition comprising exogenous PACAP into the cells, a composition of PACAP must be prepared.

The PACAP polypeptides, agonists or antagonists of the invention can be created in any suitable manner. If produced in situ, PACAP may be purified from appropriate sources, e.g., appropriate vertebrate cells e.g., mammalian cells, for instance human. Alternatively, the availability of nucleic acid molecules encoding the proteins enables production of the proteins using in vitro expression methods known in the art. For example, a PACAP cDNA or gene may be cloned into an appropriate in vitro transcription vector, for in vitro transcription, followed by cell-free translation in a suitable cell-free translation system. In vitro transcription and translation systems are commercially available, e.g., from Promega Biotech, Madison, Wis., or BRL, Rockville, Md. While in vitro transcription and translation is not the method of choice for preparing large quantities of the protein, it may be suitable for preparing small amounts of native or mutant peptides for research purposes, particularly since it allows the incorporation of radioactive nucleotides.

For the preparation of larger quantities of PACAP, the polypeptide may be produced by expression in a suitable prokaryotic or eukaryotic system. For example, part or all of a DNA molecule, such as the polynucleotide code which encodes the polypeptide of SEQ ID NO: 1, may be inserted into a plasmid vector adapted for expression in a bacterial cell (such as E. coli) or a yeast cell (such as Saccharomyces cerevisiae). Such vectors comprise the regulatory elements necessary for expression of the DNA in the host cell, positioned in such a manner as to permit expression of the DNA into the host cell. Such regulatory elements required for expression include promoter sequences, transcription initiation sequences and, optionally, enhancer sequences.

Secretion signals may be used to facilitate purification of the resulting protein. The coding sequence for the secretion peptide is operably linked to the 5′ end of the coding sequence for the protein, and this hybrid nucleic acid molecule is inserted into a plasmid adapted to express the protein in the host cell of choice. Plasmids specifically designed to express and secrete foreign proteins are available from commercial sources. For example, if expression and secretion is desired in E. coli, commonly used plasmids include pTrcPPA (Pharmacia); pPROK-C and pKK233-2 (Clontech); and pNH8a, pNH16a, pcDNAII and pAX (Stratagene), among others.

The PACAP polypeptide produced by in vitro transcription and translation or by gene expression in a recombinant prokaryotic or eukaryotic system may be purified according to methods known in the art. Recombinant peptides can be purified by affinity separation, such as by immunological interaction with antibodies that bind specifically to the peptide or fusion proteins such as His tags, as described below. Skilled practitioners commonly use such methods.

As mentioned, the protein can be produced and fused to a “tag” protein in order to facilitate subsequent purification. These fusion proteins are produced by operably-linking the nucleic acid coding sequence of the “tag” protein to the coding sequence of the peptide of interest, and expressing the fused protein by standard methods. Systems are commercially available that comprise a plasmid containing an expression cassette with the “tag” protein coding sequence and a polylinker into which a coding sequence of interest can be operably ligated. These fusion protein systems further provide chromatography matrices or beads that specifically bind the “tag” protein thereby facilitating the fusion protein purification. These fusion protein systems often have the recognition sequence of a protease at or near the junction of the “tag” protein and the protein of interest so that the “tag” protein can be removed if desired. Fusion protein systems include, but are not limited to, the His-6-tag system (Quiagen) and the glutathione-S-transferase system (Pharmacia).

More specifically, with regard to the PACAP polypeptide, using the appropriate amino acid sequence information, synthetic PACAP polypeptides, antagonists or agonist of the present invention may be prepared by various synthetic methods of peptide synthesis via condensation of one or more amino acid residues, in accordance with conventional peptide synthesis methods. Preferably, peptides are synthesized according to standard solid-phase methodologies, such as may be performed on an Applied Biosystems Model 430A peptide synthesizer (Applied Biosystems, Foster City, Calif.), according to manufacturer's instructions. Other methods of synthesizing peptides, either by solid phase methodologies or in liquid phase, are well known to those skilled in the art.

Once the composition comprising PACAP has been introduced into the cells, which may be accomplished using the methods of administration described above, the PACAP should bind to its receptor and mitosis should cease.

Alternatively, compositions comprising agonists to PACAP might also be introduced to the cerebral cortical cells, assisting the binding of PACAP to its receptors and furthering the goal of controlling the proliferation of the neurons. Agonists for PACAP that may be used include maxadilan (SEQ. ID. NO: 6), PACAP₂₇ (SEQ. ID. NO: 7), and VIP (SEQ. ID. NO: 8). Again, agonists may be administered in a research or clinical setting in the manner described herein. Alternatively, a composition comprising both PACAP and one or more agonists may be added to inhibit mitosis of the cerebral cortical cells, subventricular zone cells, hippocarnpal granule or glial cell precursors.

Another embodiment of the present invention that involves inhibiting the growth of neuronal cells using PACAP and/or related agonists may be used to combat diseases wherein the growth of neurons is uncontrolled, such as with cancer.

As will be understood by one skilled in the art, while the experimental data is largely directed toward cerebral cortical precursor cells, subventricular zone precursor cells, hippocarnpal granule or glial cell precursors are also embodied in the present invention because the cell precursors are all connected in the forebrain and have similar PACAP ligand/receptor systems. Recent research has demonstrated the relatedness of these precursor cell types. For example, it has recently been established that neurons in developing animal brains are derived from radial glial cells. (Noctor, S C 2001). Thus, due to the close relationship of all cell types discussed herein, the method of the present invention would be an effective regulator of mitosis for the referenced neuronal cell precursors and for glial cells.

### Definitions

Various terms relating to the biological molecules of the present invention are used throughout the specification and claims.

“Agonist” means a substance that has an affinity for and stimulates physiologic activity at cell receptors normally stimulated by naturally occurring substances, thus triggering a biological response.

“Antagonist” means a substance that tends to nullify the action of another, such as a substance that binds to a cell receptor without eliciting a response.

“Cell cycle” refers to the sequence of events between mitotic divisions. The cycle is conventionally divided into G0, G1, (G meaning gap), S (synthesis phase during which the DNA is replicated), G2 and M (mitosis).

“Mitosis” refers to a method of indirect division of a cell, consisting of a complex of various processes, by means of which the two daughter nuclei normally receive identical complements of the number of chromosomes characteristic of the somatic cells of the species.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the gene-encoded amino acids. “Polypeptides” include amino acid sequences modified either by natural processes, such as posttranslational processing, or by chemical modification techniques that are well known in the art. Such modifications are well described in basic texts and in more detailed monographs; as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications.

Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racernization, selenoylation, sulfation, transfer-RNA mediated addition of amino acids to proteins such as arginylation, and ubiquitination. (Creighton, W 1993).

A “conservative amino acid substitution”, as used herein, is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g.., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “substantially the same” refers to nucleic acid or amino acid sequences having sequence variation that do not materially affect the nature of the protein (i.e. the structure, stability characteristics, substrate specificity and/or biological activity of the protein). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function.

“Delivery of a therapeutic agent” may be carried out through a variety of means, such as by using parenteral delivery methods such as intravenous and subcutaneous injection, and the like. Such methods are known to those of skill in the art of drug delivery, and are further described herein in the sections regarding pharmaceutical preparations and treatment. Compositions, include pharmaceutical formulations, comprising a PACAP gene, protein, or antisense polynucleotide sequence that may be delivered by means well known in the art and described below. In such compositions, the PACAP may be in the form a DNA segment, recombinant vector or recombinant virus that is capable of expressing a PACAP protein in a cell; specifically, a cerebral cortical precursor cell. These compositions, including those comprising a recombinant viral gene delivery system, such as an adenovirus particle, may be formulated for in vivo administration by dispersion in a pharmacologically acceptable solution or buffer. Preferred pharmacologically acceptable solutions include neutral saline solutions buffered with phosphate, lactate, Tris, and the like.

The term “contacted” when applied to a cell is used herein to describe the process by which an PACAP gene or antisense sequence, and/or an PACAP polypeptide, is delivered to a target cell or is placed in direct proximity with the target cell. This delivery may be in vitro or in vivo and may involve the use of a recombinant vector system. Any method may be used to contact a cell with the PACAP associated protein or nucleotide sequence or PACAP polypeptide, so long as the method results in either increased or decreased levels of functional PACAP polypeptide within the cell or on the cell surface.

With reference to PACAP the term “contacted” includes both the direct delivery of a PACAP protein to the cell and the delivery of a gene or DNA segment that encodes PACAP, or its antisense polynucleotide sequence, which gene or antisense sequence will direct or inhibit, respectfully, the expression and production of PACAP within the cell. Since protein delivery is subject to drawbacks, such as degradation and low cellular uptake, it is contemplated that the use of a recombinant vector that expresses a PACAP protein, or encodes for an PACAP polynucleotide antisense sequence, will be of particular advantage for delivery.

“Antibodies” as used herein includes polyclonal and monoclonal antibodies, chimeric, single chain, and humanized antibodies, as well as Fab fragments, including the products of a Fab or other immunoglobulin expression library. With respect to antibodies, the term, “immunologically specific” refers to antibodies that bind to one or more epitopes of a protein of interest, but which do not substantially recognize and bind other molecules in a sample containing a mixed population of antigenic biological molecules.

The term “specific binding affinity” is meant that the antibody or antibody fragment binds to target compounds with greater affinity than it binds to other compounds under specified conditions. Antibodies or antibody fragments having specific binding affinity to a compound may be used in methods for detecting the presence and/or amount of the compound in a sample by contacting the sample with the antibody or antibody fragment under conditions such that an immunocomplex forms and detecting the presence and/or amount of the compound conjugated to the antibody or antibody fragment.

The term “polyclonal” refers to antibodies that are heterogeneous populations of antibody molecules derived from the sera of animals immunized with an antigen or an antigenic functional derivative thereof. For the production of polyclonal antibodies, various host animals may be immunized by injection with the antigen. Various adjuvants may be used to increase the immunological response, depending on the host species.

“Monoclonal antibodies” are substantially homogenous populations of antibodies to a particular antigen. They may be obtained by any technique that provides for the production of antibody molecules by continuous cell lines in culture. Monoclonal antibodies may be obtained by methods known to those skilled in the art. See, for example, U.S. Pat. No. 4,376,110.

The term “antibody fragment” refers to a portion of an antibody, often the hypervariable region and portions of the surrounding heavy and light chains, that displays specific binding affinity for a particular molecule. A hypervariable region is a portion of an antibody that physically binds to the target compound. The term “antibody fragment” also includes single charge antibodies.

With respect to “therapeutically effective amount” is an amount of the PACAP polynucleotide, PACAP antisense polynucleotide or PACAP polypeptide or protein, that when administered to a subject is effective to bring about a desired effect (e.g., an increase or decrease in precursor cell maturation, differentiation and/or proliferation) within the subject.

### EXAMPLES

The following experimental examples illustrate a working model of the PACAP ligand/receptor system in cerebral cortical precursor cells.

### Example 1

### Defining PACAP Effect on Cerebral Cortical Precursors In Vivo

To define functions in vivo, potentially responsive precursors were identified in Embryonic Day 15.5 (E15.5) rat using in situ hybridization and immunohistochemistry for PAC₁ receptor. The following methods were used:

In situ hybridization. Fresh frozen E15.5 Sprague-Dawley rat embryonic heads were sectioned at 15 μm coronally. Forty two-mer oligodeoxynucleotide probes containing the C-terminal region of PAC₁, complementary to 1342-1383 bp, were 3′-end-labeled using digoxygenin (DIG)-UTP, yielding tail lengths of ˜10 DIG-UTP. After overnight hybridization at 40° C., slides were sequentially rinsed twice for 30 min at 40° C. in 2×SSC/0.02% SDS, 1×SSC, 0.2×SSC and 0.1×SSC. Staining using NBT and X-phosphate was done according to manufacturer's instructions (Roche Molecular Biochemicals, Indianapolis, Ind.). A standard competition strategy, 50-fold excess unlabeled probe, was used as hybridization control. In addition, different oligodeoxynucleotide probes possessing similar GC content and Tms against alternative receptor isoforrns revealed no hybridization (data not shown).

Immunochemistry. For PAC₁ receptor and PACAP immunohistochemistry, frozen sections were fixed in 2% paraformaldehyde (PFA), blocked and incubated with primary antibodies (A. Arimura, Tulane University) to PAC₁ (1:500) or PACAP (1:10,000) for 2 days at 4° C. Signal was visualized using biotinylated secondary and FITC-avidin or Vectastain ABC kit (Vector Laboratories, Burlingame, Calif.). Fluorescent profiles were recorded in the line scan mode and cell profiles were obtained by DIC optics using a Bio-Rad confocal microscope.

For double labeling of mitotic cells, freshly dissociated E13.5 precursors were cultured acutely for 3 h, as described, with BrdU (10 μM, Sigma, Saint Louis, Mo.) and fixed with 4% PFA. Cultures were incubated with PACAP antibody (1:1000) for 1 h and FITC-conjugated secondary (1:200, Vector). After fixation and treatment with 2N HC1 (30 min), cultures were incubated with BrdU antibody (1:75, Becton Dickinson, San Jose, Calif.) and Texas Red-conjugated secondary (1:200, Vector). The same sequence was done with receptor antibody (1:2000), and PAC₁ and BrdU were visualized using diaminobenzidine (DAB) reaction and FITC respectively.

For P-CREB immunostaining, E15.5 heads were fixed in 4% PFA, cryoprotected, sectioned at 5 μm, incubated with primary antibody (1:2000, Upstate Biotechnology, Lake Placid, N.Y.), and visualized using DAB reaction.

For BrdU immunohistochemistry, E15.5 and E18.5 tissues were fixed in 4% PFA, paraffin-embedded, and sectioned (4 μm) coronally. E20.5 tissues were perfusion-fixed in 2% PFA, cryoprotected, and sectioned at 15 μm. The tissues were immunostained. Then, the tissues were double-labeled with antibodies to neuron specific markers TuJ1 (1:200, Biogenesis, Poole, United Kingdom), tau (1:200, I. Fischer, Med. Coll. Penn.) and MAP2c (1:2000, I. Fischer) or glial markers, vimentin (1:4, Roche) and GFAP (1:1000, Sigma), visualizing with Texas Red for BrdU and FITC for phenotypic markers. TUNEL assay was done according to the manufacturer (Oncor, Gaithersburg, Md.) on sections adjacent to those used for BrdU.

Transuterine intracerebroventricular injection. Intraventricular injection was done as described. Following anesthesia (ketamine 30 mg per kg/xylazine 3 mg per kg) and laparotomy of E15.5 pregnant dams, 1 μl of vehicle or factors and 0.05% fast green was delivered into the embryonic lateral ventricle through the uterine wall, using drawn capillary pipettes. Dye enhancement of the ventricles indicated successful injections. After closure, dams were injected subcutaneously with markers of DNA synthesis, [³H]dT (4 μCi/g, NEN) and BrdU (100 μg/g) at 3 h and sacrificed at 4 h. One embryonic hemisphere was assayed for [³H]dT incorporation and the other for immunohistochernistry.

Quantification. Newly synthesized DNA was measured after TCA precipitation. Tissues were homogenized in 10 volumes of distilled water. After reserving aliquots to assess total tissue [³H]dT uptake, DNA was precipitated with 10% TCA, centrifuged, and washed twice in 10% TCA. Pellets were dissolved and counted along with aliquots by scintillation spectrometry.

For labeling index, the analysis was done in the dorsolateral sector of 3 non-adjacent coronal cortex sections per brain, using a camera lucida (Zeiss, Thomwood, N.Y.) under 400×magnification. For E15.5 sections, BrdU(+) nuclei and total nuclei were scored within a 100 μm×100 μm sector based on the ventricular surface extending to the pial surface. BrdU(+) cells in the CP were counted in centrally located sectors extending 100 μm (E18.5 sections) or 200 μm (E20.5 sections) in the mediolateral dimension.

### Results

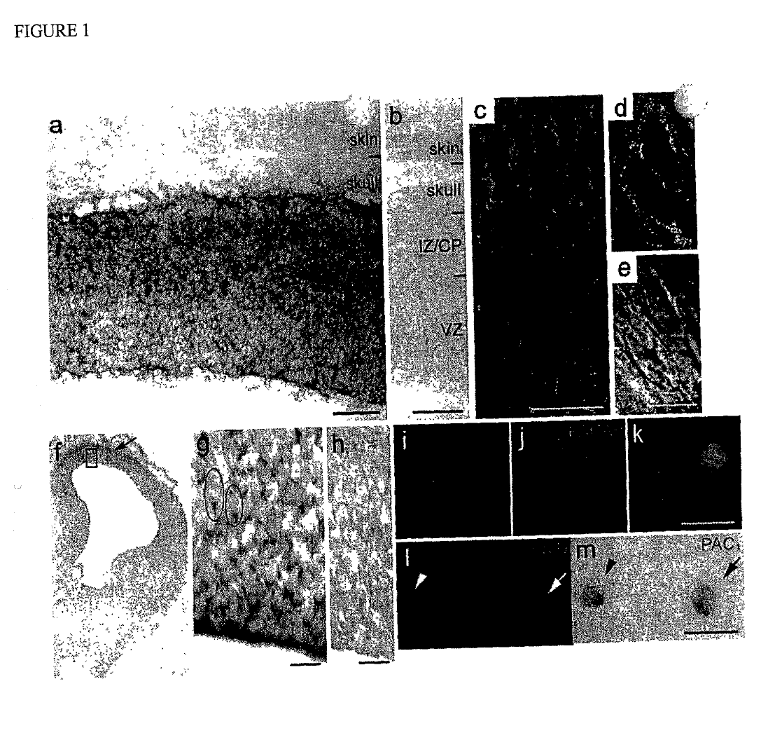

In the first example, PAC₁ transcript was detected broadly in cortex, and absent in overlying skull and skin. Transcripts were present in both proliferative ventricular zone (VZ) and post-mitotic cortical plate (CP), with more intense signal in upper VZ and intermediate zone (IZ), where precursors exit the cell cycle and begin migration and differentiation (FIG. 1a and b). FIG. 1a shows, using DIG-labeled antisense oligonucleotides, in situ localized transcripts to the brain, but not to the overlying tissues. In the competition control of FIG. 1b, no signal was detected. (Bar=50 μm).

PAC₁ receptor protein was also present in both regions, with immunoreactive signal localized to cell boundaries using laser confocal analysis (FIG. 1c-e), raising a potential role of PACAP signaling in corticogenesis. Laser confocal activity localized PAC1 immunoreactivity to cell membranes in the cortex. In FIG. 1c, cells exhibit characteristic vertical orientation in the VZ, whereas transverse section of horizontally orientated IZ cells yields round to oval profiles (upper ⅓). (Bar=20 um). In a high powered view of VZ, a fluorescent signal (as seen in FIG. 1d) is restricted to cell borders observed by DIC optics (shown in FIG. 1e). Moreover, PACAP peptide was detected, with strongest expression in the emerging preplate, composed of post-mitotic precursors. However, PACAP was also present in VZ precursors (E13.5-E14.5), appearing as cytoplasmic crescents above or below elliptical nuclei in the dense proliferative neuroepithelium. PACAP is expressed in the embryonic cortex as shown in FIGS 1f through 1h. FIG. 1f demonstrates that PACAP immunoreactivity is present primarily in the dorsal forebrain at E14.5, with prominent signals in the emerging preplate (highlighted by the arrow in the figure), overlying the VZ. The boxed region of FIG. 1f is enlarged in FIG. 1g to give an enhanced view of the region. In FIG. 1g, the intense peptide signal created by DAB in the VZ is localized to cytoplasmic crescents above or below elliptical, vertically oriented nuclei. Two representative cells are circled, the left one exhibiting a stained process extending to ventricle. Similar staining is observed at E13.5 (not shown).

### Example 2

### Defining PACAP Proliferation in Cerebral Cortical Precursors

To define relationships to proliferation, acutely dissociated precursors with mitotic S-phase marker, bromodeoxyuridine (BrdU), were labeled and double immunocytochemistry was performed. Of precursors in S-phase, representing 25±1.5% of the population (Lu, N 1997), over 95% expressed PACAP and PAC_{1,} receptor (FIG. 1 i-m), because peptide signaling influences proliferation in vivo. FIGS. 1i-k show the co-localization of BrdU and PACAP, with Texas Red (FIG. 1i), PACAP with FITC (FIG. 1j) and a combined image (FIG. 1k). FIGS. 1l-m show co-localization of BrdU and the PAC1 receptor. FIG. 1l is a visualization of BrdU with FITC and PAC₁. FIG. 1m is a visualization with a DAB reaction, with the arrow indicating the BrdU(+)/PAC₁ (+) cell and the arrowhead indicating BrdU(−)/PAC₁(+) cell. (Bar=10 μm). Furthermore, PACAP expression by single precursors corroborates previous evidence of MRNA and protein in cortical tissue population (Lu, N 1997; Waschek, J A 1998; Tatsuno, I 1994; Sheward, W J 1996), suggesting an autocrine/paracrine role for PACAP in vivo, as observed in culture population (Lu, N 1997).

### Example 3

### Defining PACAP Function in Embryos

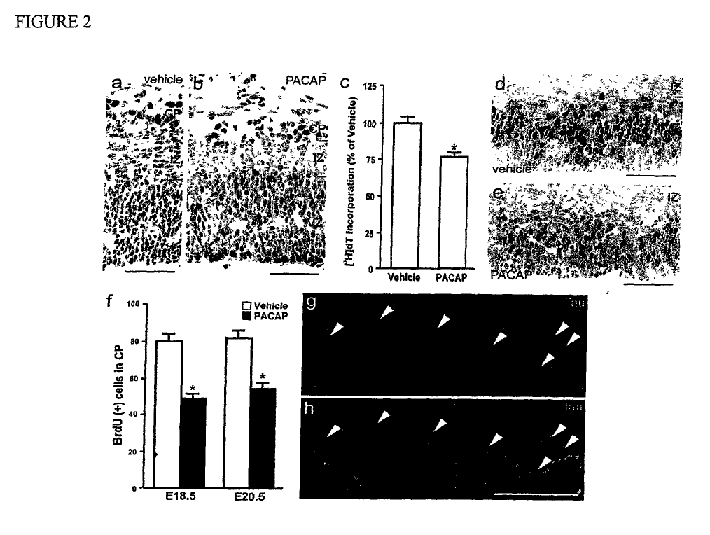

To define functions in embryos, PACAP (10⁻⁵M) was administered by transuterine intracerebroventricular injection (ICV) into lateral ventricles of E15.5 rat brains. The experiments determined that intraventricular PACAP inhibits precursor mitosis and neurogenesis without enhancing cell death (FIG. 2) As PAC₁ is expressed broadly, responsive cells were analyzed by nuclear P-CREB immunoreactivity, a downstream signaling pathway defined in vitro population (Lu, N 1997). Five minutes after PACAP injection, P-CREB signal was observed in many nuclei in the VZ nuclei (FIG. 2b) , but not in the vehicle-treated IZ nuclei (FIG. 2a), indicating that a subset of VZ precursors express rapidly responsive PAC_{1,} receptors. This signal was brief, however, no longer detected after 30 minutes (not shown). In both vehicle (FIG. 2a) and PACAP treated samples, P-CREB signals were present in the CP. Four hours later, DNA synthesis, measured by assaying [³H]thymidine ([³H]dT) incorporation into cerebral hemispheres, was decreased 24% (FIG. 2c), suggesting that PACAP signaling inhibits mitogenesis in vivo. The data are presented as a mean percent of control ±SEM from six independent experiments (vehicle-injected embryos, n=25; PACAP-injected embryos, n=27). *, P<0.0003. Vehicle incorporation=526 cpm.

The PACAP-induced reduction in DNA synthesis may result from decreased cell entry into mitotic S-phase or enhanced cell death. To estimate cells entering S-phase (Labeling Index, LI) in the VZ (Takahashi, T 1996), BrdU immunohistochemistry was performed at 4 hours (FIG. 2d, 2e). PACAP decreased the LI by 26% (LI; vehicle=36±0.3%; PACAP=26±1.3%, n=6/group, P<0.0001), comparable to effects on DNA synthesis. Thus, PACAP decreased the proportion of precursors in the mitotic cycle, visualized as BrdU-immunoreactive cells in the VZ. Bar=50μm. In contrast, TUNEL analysis of apoptosis detected no difference (vehicle, 0.5±0.4 nuclei/section; PACAP, 0.2±0.1 nuclei/section; P>0.05; n=4/group), indicating that PACAP signaling is sufficient to inhibit precursors from entering mitotic S-phase.

### Example 4

### Defining the Effects of PACAP Regulation During Mitosis to Change the Complement of Cells Migrating to the CP

Acute PACAP regulation of mitosis may alter the complement of cells subsequently migrating to the CP. To begin defining effects, BrdU-labeled precursors in the CP at two later stages following PACAP treatment at E15.5. The effects of PACAP at E18.5 and E20.5 were estimated. BrdU(+) cells in the CP were counted 3 days (vehicle, n=5; PACAP, n=5) and 5 days (vehicle, n=4; PACAP, n=5) after PACAP treatment. *, P<0.0001. Nuclear size of cells did not differ between groups (not shown).

There were 38% and 33% less mitotically labeled cells at 3 (E18.5) days and 5 (E20.5) days after PACAP administration (FIG. 2f), suggesting that inhibiting precursor proliferation affects final population size. PACAP likely affects cells in the neuronal lineage. Another experiment indicates that BrdU-labeled nuclei colocalize with neuronal markers, tau and neuron-specific class III β tubulin in the CP (FIG. 2g-2h), but not with glial markers, vimentin or GFAP, suggesting that peptide treatment did not affect gliogenesis nor alter the fate of CP precursors. FIGS. 2g and 2h show VZ precursors labeled with BrdU at E15.5 exhibit neuronal markers at later stages. In FIG. 2g, CP cells at E18.5 exhibit intense cytoplasmic signal for neuronal filament, tau (FITC) following PACAP treatment. FIG. 2g displays 4μm sections. Arrowheads indicate absent signal in nuclei. FIG. 2h shows a double immunocytochemistry assay demonstrating BrdU labeled (rhodamine) nuclei surrounded by tau cytoplasm in CP. (Bar=50μm.)

Since PACAP receptor activation increased cAMP in vitro population (Lu, N 1997), and elicited P-CREB expression in vivo (FIG. 2), cAMP signaling was then assessed using non-metabolizable analog, 8-bromo-cAMP. A robust 33% reduction of DNA synthesis was observed, suggesting that cAMP pathways are involved in mitogenic inhibition in the embryo (Vehicle=283cpm; vehicle, n=13; 8-Br-cAMP, n=16; P<0.0001).

The overlapping expression of PACAP and PAC₁, and inhibitory effects of exogenous peptide and cAMP probably shows an autocrine/paracrine circuit whereby PACAP signaling tonically restrains ongoing mitosis. To further investigate this issue, three alternative approaches in vivo were used. First, the specific PAC₁, antagonist, PACAP₆₋₃₈, was injected to interrupt endogenous PACAP activity. The antagonist stimulated [³H]dT incorporation by 10±2.2% (Vehicle=686 cpm; vehicle, n=16; PACAP₆₋₃₈, n=15; P<0.0007), suggesting that PACAP actively inhibits ongoing proliferation. However, these effects were small, potentially due to brief duration of action (PACAP increased P-CREB at 5 min, but not 30 min), consequent to rapid cerebrospinal fluid efflux or local peptide metabolism. To enhance effects, we used a newly defined PAC₁, antagonist (Max.d.4/M65) possessing 6-fold higher affinity and greater potency than PACAP₆₋₃₈, derived from sand fly peptide maxadilan, which exhibits little sequence homology but similar secondary structure (Moro, O 1999). M65 stimulated DNA synthesis by 14% at 1 μ1 of injectant, and stimulated DNA synthesis by 20% at twice the dose (Table 1). Mitogenic stimulation elicited by two entirely unrelated PAC₁, antagonists provides strong evidence of PACAP anti-mitogenic signaling in vivo. Finally, the cAMP pathway was blocked by injecting non-metabolizable antagonist that induced a 28% increase in DNA synthesis (Table 1), consistent with a role for the G-protein coupled receptor linked to cAMP in ongoing anti-mitogenic signaling.

TABLE 1
Effect of antagonists to FAGS (M65) and cAMP (Rp-cAMPS) on DNA
synthesis

| | Injectant | Number of Animals | | |
|---|---|---|---|---|
| Reagent | Volume (μl) | Vehicle | Experimental | % Stimulation |
| M65 | 1 | 11 | 12 | 13.4 ± 4.6* |
| M65 | 2 | 6 | 6 | 20.1 ± 7.0* |
| Rp-cAMPS | 1 | 11 | 11 | 28.3 ± 3.1** |

*P<0.05, **P<0.0001

The results demonstrate that endogenous, inhibitory signals are involved in regulating precursor proliferation in the developing central nervous system in vivo, consistent with interactions among positive and negative factors observed in diverse tissues. In the skeletal system, when the TGFβ superfamily member, myostatin, was disrupted, muscles grew three times their normal size, containing more cells (McPherron, A C 1997). In the immune system, blocking the inhibitor of hematopoietic stem cell proliferation, MIP-1α, elicited marked expansion of B cells and neutrophils, and myeloid hyperplasia (Cacalano, G 1994), suggesting that inhibitory signals normally restrain proliferative expansion of precursor pools. Similarly, the PACAP and cAMP systems likely interact with endogenous cortical and other neuronal or glial mitogens during neurogenesis, as their expression overlaps with that of stimulatory factors, and correlate precisely with the time that precursors exit the cell cycle to generate the first post-mitonic neurons. Indeed, PACAP inhibited proliferative effects of mitogenic IGF-I, bFGF, and EGF in culture population (Lu, N 1997), whereas in vivo, blocking PACAP and cAMP anti-mitogenic functions yielded enhanced precursor mitosis.

Two approaches were used to define the role of PACAP in vivo, including injecting exogenous peptide and interrupting ongoing PAC₁ receptor signaling. PACAP treatment elicited a rapid P-CREB response in a subset of VZ precursors at 5 minutes, and blocked mitosis in a comparable proportion 4 hours later. The 25% reduction in mitotic labeling conforms to expectations for an anti-mitogenic signal acting at most 3-4 hours on cells with a cycle time of 11.4˜15.1 hours (Takahashi, T 1996) since only one quarter of the population crosses the G₁/S boundary during this interval. Any greater effect would raise concerns regarding a toxic response or actions on cell cycle stages other than G₁. Conversely, blockade of PAC₁, receptor and cAMP signaling elicited 20-30% more DNA synthesis in 4 hrs, consistent with S-phase re-entry by the subpopulation of precursors actually exiting the mitotic cycle at this age, approximately 19˜34% (Takahashi, T 1996, see Table 3).

While effects of PACAP were brief, one third less BrdU-labeled cells were in the CP 3 and 5 days later, suggesting that endogenous anti-mitogenic signaling in the VZ may impact on ultimate corticogenesis. Mathematical modeling suggests that neurogenetic amplification is determined by the proliferative (P) and quiescent/output (Q) fractions during a fixed number of cell cycles (Takahashi, T 1996). If PACAP reduces the P fraction at a given time point, the generated population will be diminished with each succeeding cycle. Further, consistent with classical studies, altering neurogenesis at E15.5 affected generation of neurons.

PACAP is an important anti-mitogenic signal during ontogeny, and studies in vitro have identified other secreted cortical factors eliciting inhibition, including glutamate and GABA (Lo Turro, J J 1995; Antonopoulos, J 1997). Potentially, different signals, alone or in combination, act on distinct precursor populations, consistent with interactions of PACAP and glutamate in regulating cortical neuron arachidonic acid release and c-fos and BDNF expression (Pellegri, G 1998). Furthermore, while PACAP and cAMP effects were quite comparable, these studies imply that other cAMP-linked pathways may impact proliferation, including dopamine, somatostatin, and serotonin, of particular concern as targets of current potent therapies.

More generally, an array of negative signals, originating from the proliferative zone or the post-mitotic cortical plate, interacts with positive factors to regulate precursor proliferation and neurogenesis. This model system will be useful for analyzing extracellular signals, and defining cell cycle regulation in the forebrain of the developing embryo.

The foregoing is intended to be illustrative of the embodiments of the present invention, and is not intended to limit the invention in any way. Although the invention has been described with respect to specific modifications, the details thereof are not to be construed as limitations, for it will be apparent to those skilled in the art that various equivalents, changes and modifications may be resorted to without departing from the spirit and scope thereof and it is understood that such equivalent embodiments are to be inherently included herein.

All publications and patent applications are herein incorporated by reference to the same extent as if each individual publication or patent application was specifically and individually indicated to be incorporated by reference.

### REFERENCES CITED

Antonopoulos, J., Pappas, 1. S. & Pamavelas, J. G. Activation of the GABAA receptor inhibits the proliferative effects of bFGF in corticalprogenitor cells. EUR J NEUROSCI 9, 291-298 (1997).

Arimura A., Perspectives on pituitary adenylate cyclase activating polypeptide (PA CAP) in the neuroendocrine, endocrine, and nervous systems, JPN J PHYSIO 48: 301-331 (1998).

Cacalano, G. et al. Neutrophil and B cell expansion in mice that lack the murine IL-8 receptor homolog. SCIENCE 265, 682-684(1994).

Chattedee T K, Sharma R V, Fisher R A, Molecule cloning of a novel variant of the pituitary adenylate cyclase activating polypeptide (PACAP) receptor that stimulates calcium influx by activation ofL-type calcium channels, J BIOL CHEM 271: 32226-32232 (1996).

Cole, et al., MONOCLONAL ANTIBODIES AND CANCER THERAPY pp. 77-96 (1985).

Creighton, T. E., W. H. Freeman and Company, Proteins-Structure And Molecular Properties, 2nd Ed., New York, 1993.

DiCicco-Bloom, E. et al. Autocrine expression and ontogenetic functions of the PACAP ligand/receptor system during sympathetic development. DEV BIOL 219, 197-213 (2000).

Drago, J., Murphy, M., Carroll, S. M., Harvey, R. P. & Bartlett, P. F. Fibroblast growth factor mediatedproliferation of central nervous system precursors depends on endogenous production of insulin-like growth factor I. PROC NATL ACAD SCI U S A 88, 2199-2203 (1991).

Ghosh, A. & Greenberg, M. E. Distinct roles for bFGF and NT-3 in the regulation of cortical neurogenesis. NEURON 15, 89-103 (1995).

Harlow and Lane, Antibodies; A Laboratory Manual, COLD SPRINGS HARBOR LABORATORY (1988).

Kohler and Milstein, NATURE 256:495-497 (1975).

Kozbor et al., IMMUNOLOGY TODAY 4:72 (1983).

LoTurco, J. J., Owens, D. F., Heath, M. J., Davis, M. B. & Kriegstein, A. R. GABA an glutamate depolarize corticalprogenitor cells and inhibit DNA synthesis. NEURON 15, 1287-1298 (1995).

Lu, N. & DiCicco-Bloom, E. Pituitary adenylate cyclase-activatingpolypeptide is an autocrine inhibitor of mitosis in cultured corticalprecursor cells. PROC NATL ACAD SCI U S A 94, 3357-3362 (1997).

McPherron, A. C., Lawler, A. M. & Lee, S. J. Regulation of skeletal muscle mass in mice by new TGF-beta superfamily member. NATURE 387, 83-90 (1997).

Miyata A, Arimura A, Dahl R R, Minamino N, Uehara A, Jiang L, Culler M D, Coy DH: Isolation of a novel 38 residue-hypothalamic polypeptide which stimulates adenylate cyclase in pituitary cells, BIOCHEM BIOPHYS RES COMMUN 164: 567-74, (1989).

Moro, O. et al. Functional characterization ofstructural alterations in the sequence of the vasodilatory peptide Maxadilan yields a Pituitary Adenylate Cyclase-activating Peptide type 1 receptor-specific antagonist J BIOL CHEM 274, 23103-23110 (1999).

Nicot A, DiCicco-Bloom E, Regulation ofneuroblast mitosis is determined by PA CAP receptor isoformexpression, PROC NATL ACAD SCI USA 98: 4758-4763 (2001).

Noctor, S. C., Flint, A. C., Weissman, T. A., Dammerman, R. S., & Kriegstein, A. R., Neurons derived from radial glial cells establish radial units in neocortex, NATURE, 409: 714-720 (2001).

Pellegri, G., Magistretti, P. J. & Martin, J. L. VIP and PACAP potentiate the action of glutamate on BDNF expression in mouse cortical neurones. EUR J NEUROSCI 10, 272-280 (1998).

Sheward, W. J., Lutz, E. M. & Harnar, A. J. Expression ofpituitary adenylate cyclase activating polypeptide receptors in the early mouse embryo as assessed by reverse transcription polymerase chain reaction and in situ hybridisation. NEUROSCI LETT 216, 45-48 (1996).

Spengler D, Weaber C, Pantaloni C, Holsboer F, Bockaert J, Seeburg P H, Joumot L, Differential signal transduction by five splice variants of the PACAP receptor, NATURE 365: 170-175 (1993).

Suh, Junghyup, Nairu Lu, Arnaud Nicot, Ichiro Tatsuno, Emanuel DiCicco-Bloom, PACAP is an anti-mitogenic signal in developing cerebral cortex, NATURE NEUROSCIENCE, 4: 123-124 (2001).

Takahashi, T., Nowakowski, R. S., & Caviness, V. S., The leaving or Qfraction of the murine cerebral proliferative epithelium: a general model of neocortical neurogenesis, JNEUROSCI. 16: 6183-6196 (1996).

Tatsuno, I., Somogyvari-Vigh, A. & Arimura, A. Developmental changes ofpituitary adenylate cyclase activating polypeptide (PACAP) and its receptor in the rat brain. PEPTIDES 15, 55-60 (1994).

Vaccarino, F. M. et al. Changes in cerebral cortex size are governed byfibroblast growth facto during embryogenesis. NAT NEUROSCI 2, 246-253 (1999).

Waschek, J. A. et al. Neural tube expression ofpituitary adenylate cyclase-activating peptide (PACAP) and receptor: potential role in patterning and neurogenesis. PROC NATL ACAD SCI U S A 95, 9602-9607 (1998).

1. A method of proliferating neuronal precursor cells, comprising contacting the precursor cells with an antagonist for a pituitary adenylate cyclase activating polypeptide (PACAP) receptor, PAC₁.

2. The method of claim 1, wherein the neuronal precursor cells are cerebral cortical precursor cells, subventricular zone precursor cells, or hippocampal granule precursor cells.

3. The method of claim 1, wherein the neuronal precursor cells are cerebral cortical precursor cells.

4. The method of claim 1, wherein PACi has an amino acid sequence of SEQ ID No: 3:

5. The method of claim 1, wherein the antagonist is PACAP₆₋₃₈ .

6. The method of claim 5, wherein the antagonist has an amino acid sequence of SEQ ID NO: 4.

7. The method of claim 1, wherein the antagonist is max d 4.

8. The method of claim 7, wherein the antagonist has an amino acid sequence of SEQ ID NO: 5.

9. The method of claim 1, wherein the antagonist is non-metabolizable.

10. A method of inhibiting proliferation of neuronal precursor cells, comprising contacting the cells with a composition comprising pituitary adenylate cyclase-activating polypeptide (PACAP).

11. The method of claim 10, wherein the neuronal precursor cells are cerebral cortical precursor cells, subventricular zone precursor cells, or hippocampal granule precursor cells.

12. The method of claim 10, wherein the neuronal precursor cells are cerebral cortical precursor cells.

13. The method of claim 10, wherein PACAP has a nucleotide sequence of SEQ ID NO: 1 or an amino acid sequence of SEQ ID NO: 2.

14. The method of claim 10, further comprising adding an agonist for a PACAP receptor, PAC₁.

15. The method of claim 10, wherein PAC₁ has an amino acid sequence of SEQ ID NO: 3.

16. The method of claim 10, wherein the agonist is maxadilan.

17. The method of claim 16, wherein maxadilan has an amino acid sequence of SEQ ID NO: 6.

18. The method of claim 10, wherein the agonist is PACAP₂₇.

19. The method of claim 18, wherein PACAP₂₇ has an amino acid sequence of SEQ ID NO: 7.

20. The method of claim 10, wherein the agonist is VIP.

21. The method of claim 20, wherein VIP has an amino acid sequence of SEQ ID NO: 8.

22. The method of claim 10, wherein the composition comprising PACAP passes through the blood-brain barrier.

23. The method of claim 10, wherein the composition reduces DNA synthesis.

24. The method of claim 10, wherein inhibiting proliferation of the cells comprises inhibiting mitosis of the cells.

25. The method of claim 24, wherein inhibiting mitosis comprises blocking the G1-S phase transition in the cell development.

26. A method of promoting proliferation of neuronal precursor cells, comprising
providing an oligonucleotide consisting of a sequence complementary to PACAP; and
introducing the oligonucleotide into the cells, wherein the oligonucleotide decreases the expression of PACAP in the cells.

27. The method of claim 26, wherein the neuronal precursor cells are cerebral cortical precursor cells, subventricular zone precursor cells, or hippocampal granule precursor cells.

28. The method of claim 26, wherein the neuronal precursor cells are cerebral cortical precursor cells.

29. The method of claim 26, wherein the oligonucleotide is DNA, cDNA, RNA, or nMRNA.

30. A method of promoting proliferation of neuronal precursor cells, comprising
providing an antibody that binds to PACAP; and
introducing the antibody into the cells, wherein the antibody decreases expression of PACAP in the cells.

31. The method of claim 30, wherein the neuronal precursor cells are cerebral cortical precursor cells, subventricular zone precursor cells, or hippocampal granule precursor cells.

32. The method of claim 30, wherein the neuronal precursor cells are cerebral cortical precursor cells.

33. A method of treating a medical condition caused by the proliferation of neuronal precursor cells, comprising administering a composition comprising PACAP to said cells.

34. The method of claim 33, wherein the neuronal precursor cells are cerebral cortical precursor cells, subventricular zone precursor cells, or hippocampal granule or glial precursor cells.

35. The method of claim 33, wherein the neuronal precursor cells are cerebral cortical precursor cells.

36. The method of claim 33, wherein PACAP has a nucleotide sequence of SEQ ID NO: 1 or an amino acid sequence of SEQ ID NO: 2.

37. The method of claim 33, wherein cell growth is inhibited.

38. The method of claim 37, wherein the cells are cancerous.

39. A method of treating a medical condition caused by the loss of neuronal precursor cells, comprising administering an antagonist for PAC₁ receptor to the cells.

40. The method of claim 39, wherein the neuronal precursor cells are cerebral cortical precursor cells, subventricular zone precursor cells, or hippocampal granule precursor cells.

41. The method of claim 39, wherein the neuronal precursor cells are cerebral cortical precursor cells.

42. The method of claim 39, wherein the PAC₁ receptor has an amino acid sequence of SEQ ID NO: 3.

43. The method of claim 39, wherein neuronal cell growth is promoted.

44. The method of claim 39, wherein the medical condition is a stroke, dementia, primary cortical degenerative disorders, sub-cortical degenerative disorders, infections, prion disorders, toxic and metabolic disorders, and brain injury.

45. A method of increasing brain tissue, comprising administering a PACAP antagonist to neuronal precursor cells.
